(19) United States
(12) Patent Application Publication
(10) Pub. No.: US 2007/0049801 A1
(43) Pub. Date: Mar. 1, 2007

(54) Endoscope accessory

(76) Inventors: Ronald B. Lamport, Pelham, NH (US); James Loper, Wales, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/318,086

(22) Filed: Dec. 22, 2005

Related U.S. Application Data
(60) Provisional application No. 60/711,000, filed on Aug. 24, 2005.

(51) Int. Cl. A61B 1/12

(52) U.S. Cl. 600/154; 600/159

(57) ABSTRACT

An endoscope accessory capable of being removably coupled to the accessory port of an endoscope includes a housing having an introducer port adapted for insertion of an elongated member, a device port adapted for alignment with the accessory port, a fluid port in fluid communication with the device port, and a fluid-tight seal, which forms a seal around the elongated member. The accessory includes a coupling mechanism to couple the accessory to the accessory port to assist in an endoscopic procedure. For example, the elongated member can be inserted through the introducer port, into the accessory port with which the device port is aligned, and into a patient's gastrointestinal tract. The patient's tissue may be irrigated by injecting fluid through the fluid port while the elongated member is manipulated. A locking mechanism, coupled to the housing, can be used to secure the elongated member with respect to the endoscope.

### RELATED APPLICATION

This application claims the benefit of U.S. Provisional Application No. 60/711,000 filed Aug. 24, 2005. The entire teachings of the above application(s) are incorporated herein by reference.

### BACKGROUND OF THE INVENTION

Endoscopic treatments for treating abnormal pathologies, as well as for treatment of obesity, diabetes and other ailments are increasing in number. The endoscope provides access to the patient's gastrointestinal tract through natural orifices and permits direct visualization of tissues.

The proximal end of the endoscope typically has an accessory and/or irrigation port through which accessories, such as catheters and guidewires, can be inserted or fluid may be injected. The accessory port leads into the accessory channel of the endoscope with an opening at its distal end through which the catheter, guidewire or fluid can extend into the patient.

To maintain the position of the guidewire or catheter with respect to the endoscope, the physician may need to maneuver the catheter or guidewire with one hand, and the endoscope with the other hand. External clips can be used for securing the guide wire or catheter to the endoscope. Further, the physician may also need to irrigate the tissues with fluid though the accessory channel while a catheter or guidewire is in place.

### SUMMARY OF THE INVENTION

The present disclosure is directed to an endoscope accessory capable of being attached to the proximal portion of a medical instrument, such as an endoscope, for use during a medical procedure. The endoscope accessory includes a housing defining a lumen. The lumen extends between a proximal introducer port and a distal device port of the housing, and is adapted to have an elongated member inserted through it. The endoscope accessory further includes a normally closed valve or fluid-tight seal which is adapted to form a seal around the elongated member inserted through the endoscope accessory. The fluid-tight seal also prohibits the flow of fluid through the introducer port on the endoscope accessory. The endoscope accessory also includes a coupling mechanism coupled to the housing, which is adapted to removably couple the endoscope accessory to a proximal portion of a medical instrument. The endoscope accessory further includes a locking mechanism coupled to the housing. The endoscope accessory also includes a fluid port, which is in fluid communication with the lumen. The fluid port is adapted to permit the injection of a fluid through it to the device port.

The locking mechanism secures the elongated member with respect to the housing. The locking mechanism can be slidably engaged with the housing. The locking mechanism can, for example, be a pinch clamp.

The coupling mechanism has a sleeve and a shoulder to abut a flange on the accessory port of the endoscope accessory. The coupling mechanism also forms a fluid tight seal with the endoscope accessory port, such that fluid does not leak from the endoscope accessory when it is attached to the endoscope accessory port. The coupling mechanism is threaded onto the housing.

The device port on the housing is adapted for alignment with an introducer port disposed upon the proximal end of the medical instrument. For example, the accessory port may be the introducer port of the endoscope.

The fluid port is in fluid communication with the device port. The fluid port permits the injection of fluid alongside the elongated member, or a catheter.

The fluid-tight seal is adapted to prohibit fluid communication between the fluid port and the introducer port. Thus, the spilling of fluid through the introducer port is prevented while the fluid is channeled from the fluid port into the attached medical instrument. The fluid-tight seal is preferably a duck-bill valve.

The endoscope accessory can further include a valve coupled to the housing. The valve can be used for regulating fluid flow through the fluid port and into at least a portion of the housing.

The present disclosure is further directed to a method of performing an endoscopic procedure using the endoscope accessory. The steps include coupling an endoscope accessory to an accessory port of an endoscope. The method further includes inserting at least a portion of an elongated member into a lumen within a patient's body via an accessory port of the endoscope. The method also includes irrigating the lumen within the patient's body with fluid injected through the accessory port while the elongated member is inserted within the accessory port. Further, the method includes securing the elongated member against axial movement with respect to the endoscope accessory using a locking mechanism, coupled to the endoscope accessory.

Coupling the endoscope accessory includes using a coupling mechanism. The coupling mechanism includes a sleeve having a shoulder, which abuts a proximal flange on the accessory port of the endoscope.

In one embodiment, inserting the elongated member through the accessory port of the endoscope further includes inserting the elongated member into an introducer port of the endoscope accessory, where the endoscope accessory is coupled to the accessory port of the endoscope.

For example, the endoscopic procedure can be the placement of a gastrointestinal liner within a patient's gastrointestinal tract. As the stent or gastrointestinal liner is placed or repositioned within the gastrointestinal tract, the endoscopist may choose to irrigate the patient's gastrointestinal tract. Such irrigation facilitates the placement of the gastrointestinal sleeve and improves visualization through the endoscope.

Beneficially, the endoscopist can secure the elongated member with respect to the endoscope at any time during the endoscopic procedure. This may be accomplished by using the pinch clamp locking mechanism of the endoscope accessory. At least one advantage of securing the elongated member is that the elongated member no longer needs to be maneuvered by the endoscopist, thus freeing his/her hands to perform alternative steps in the medical procedure. Further, the securing of the elongated member allows both the endoscope and the elongated member to be removed from the body together without using a manual hold and without slipping.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a perspective view of an embodiment of the invention;

FIG. 2 shows a perspective cross section of an embodiment of the invention;

FIG. 3 shows further detail of a locking mechanism;

FIG. 4 shows further detail of a locking mechanism positioned within the invention;

FIG. 5 shows the embodiment of FIG. 2 coupled to an endoscope;

FIG. 6 shows a perspective view of the embodiment in FIG. 1, as coupled to an endoscope; and

FIG. 7 shows further detail of a coupling mechanism of the invention.

### DETAILED DESCRIPTION OF THE INVENTION

A description of preferred embodiments of the invention follows.

In some medical procedures, such as the repositioning of a gastrointestinal implant within a patient's gastrointestinal tract, manipulation of an endoscope, maneuvering of a grasper used for repositioning an implant or other foreign body and irrigation of the gastrointestinal tract may be required simultaneously. Gastrointestinal implants can be used for a number of treatments, at least some of which are described in U.S. patent application Ser. No. 10/339,786, filed on Jan. 9, 2003, and claiming the benefit of U.S. Provisional Application No. 60/430,321, filed on Dec. 2, 2002, and incorporated herein by reference in their entirety. The manipulation of several medical instruments is not only difficult for the physicians or operator performing the procedure, but may pose the risk of injury to the patient.

The endoscope accessory facilitates the physician's performance of a medical procedure by combining several functions in one instrument and removing additional manual manipulation that would otherwise be needed.

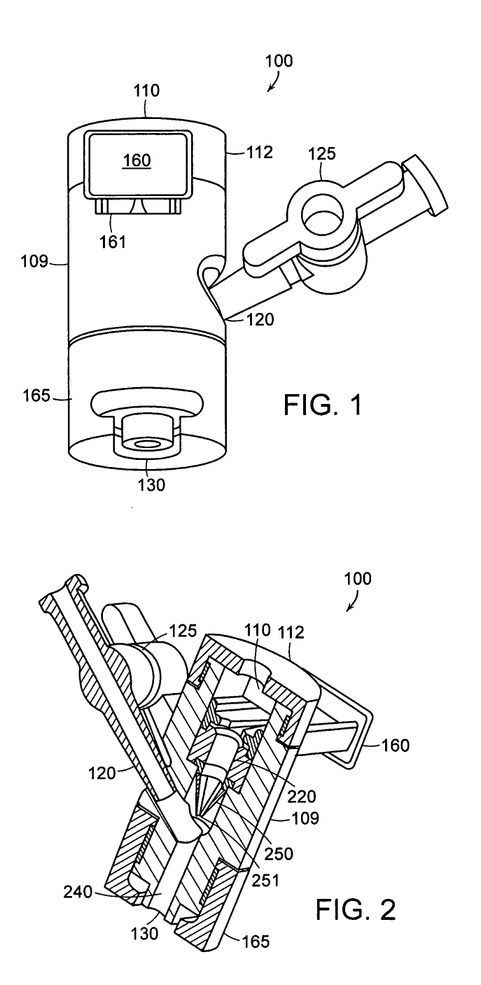

FIG. 1 shows a perspective view of an endoscope accessory 100. The endoscope accessory 100 includes housing 109 having an introducer port 110. The introducer port 110 is a hole that is located at a proximal end of the housing 109 and is adapted for insertion of an elongated member. The elongated member may include a retrieval grasper, snare, guidewire or injection catheter. In particular, the elongated member can include any means capable of grasping.

The introducer port 110 is located in a cap 112. The cap 112 can be threaded onto the housing 109. Alternatively, the cap 112 can be attached to the housing 109 using any suitable means of attachment, such as adhesive or an interference or compression fit.

The housing 109 further includes a fluid port 120. The fluid port 120 is adapted to have fluid injected through it. The fluid port 120 may be connected to an irrigation source, such as a syringe, fluid pump or gravity reservoir through a tube in order to provide fluid for irrigation.

The housing 109 also includes a device port 130. The device port 130 is located on a distal end of the housing 109. The device port 130 is adapted to accept an elongated member being inserted or withdrawn through it. Also, the device port 130 is adapted to align and provide a fluid seal between the endoscope accessory 100 and a proximal opening of the separate medical instrument.

The endoscope accessory 100 further includes a locking mechanism 160, adapted to secure the elongated member with respect to the housing 109 and thus the medical instrument during a medical procedure. The locking mechanism 160 is capable of being secured into place upon the housing 109 by sliding the locking mechanism 160 in a slot 161. The slot 161 is located between the cap 112 containing the introducer port 110 and the housing 109.

The endoscope accessory 100 may further include a valve 125. The valve 125 may be used to regulate the flow of the injected fluid through the fluid port 120. The valve 125 may be, for example, a stopcock, ball valve, gate valve, pin valve or any other type of valve capable of regulating fluid flow.

The valve 125 may be adjusted manually by an operator. The valve may be attached to the endoscope accessory 100 using an interference fit, a clamp, a thread or any other suitable attachment means. Alternatively, the valve 125 may be formed within the housing 109 of the endoscope accessory 100.

The endoscope accessory 100 includes a coupling mechanism 165. The coupling mechanism 165 may be threaded onto the distal end of the housing 109 containing the device port 130. The coupling mechanism 165 facilitates the coupling of the endoscope accessory 100 to the proximal portion of the medical instrument, such as the accessory port of the endoscope. The coupling mechanism 165 serves to mate with the medical instrument through which catheter or fluid injection is desired, such as the accessory channel of the endoscope.

The endoscope accessory 100 may be cylindrical in shape, but may be any shape capable of fitting onto the proximal portion of the medical instrument. The endoscope accessory 100 can be rigid, and may be made of any suitable rigid material, such as plastic, glass, ceramic, metal, alloys or composites. The rigidness of the endoscope accessory 100 assures the appropriate positioning of the introducer port 110, the device port 130 and the fluid port 120 during the medical procedure.

A cross section of the endoscope accessory 100 is shown in FIG. 2. The housing 109 defines a lumen 240 coupled between the introducer port 110 and the device port 130 of the endoscope accessory 100. The elongated member can slide within the lumen 240. Fluid injected into the fluid port 120 flows into at least a portion of the lumen 240. Preferably, the fluid injected through the fluid port 120 flows through the device port 130 and into the separate medical instrument.

The elongated member can be inserted through the introducer port 110. The elongated member extends distally through the lumen 140, further extending distally through the device port 130 and into the introducer port disposed upon another medical instrument, such as the accessory port of a standard endoscope.

Irrigation through the fluid port 120 may occur while the elongated member is being advanced distally through the device port 130. Irrigation during the advancement of the elongated member may be advantageous in conducting a medical procedure. For example, if the medical procedure is the placement of a gastrointestinal liner (implant) in the gastrointestinal tract of a patient's body, irrigation of the gastrointestinal tract may facilitate the distal advancement of the implant therein which is coupled to the elongated member by providing a consistent distal flow of fluid. Additionally, the pressure of the fluid in the gastrointestinal tract may cause radial expansion of the gastrointestinal tract which provides an expanded space for the implant to be advanced. This may prevent damage to surrounding tissue which would otherwise be caused by dragging an implant through an unexpanded gastrointestinal tract. The irrigation of a lumen within a patient's body while extending an elongated member through the lumen may be advantageous in several alternative medical procedures.

The endoscope accessory 100 may further include a fluid-tight seal 250. When the elongated member is advanced through the lumen 240, the fluid-tight seal 250 forms a seal around the elongated member, thus prohibiting fluid from flowing between the fluid port 120 and out of the introducer port 110 when the elongated member is inserted therein. Preferably, the fluid-tight seal 250 is capable of allowing the advancement and/or withdrawal of the elongated member through it, while forming a seal around the elongated member. The fluid-tight seal 250 is disposed within the lumen 240, between the introducer port 110 and the fluid port 120.

The fluid tight seal 250 is preferably a duckbill valve with flaps 251, which are composed of rubber. The duckbill valve 250 can be held in place using a valve retainer 220. Alternatively the duckbill valve 250 can be manually inserted and fit within the housing 109 using an interference fit or bonded to the interior of the housing 109 or attached by any suitable attachment means.

When the elongated member is pushed through the duckbill valve 250, the force exerted on the flaps 251 causes them to open allowing the advancement of the elongated member through the flaps, while forming a fluid seal around the elongated member. Thus, fluid flow proximal to the fluid tight seal 250 within the housing 109 is prevented.

Alternatively, the fluid-tight seal 250 can be a thin elastomeric disk made from silicon. The silicon disk simply contains a perforation, and has no material removed. When the elongated member is pushed through it, stress will cause it to open enough to let the elongated member through, while forming a tight seal around it. The silicon disk therefore stretches elastically but does not deform plastically. This is advantageous in that the operator must not actively perform additional steps in order to seal the fluid. Thus, fluid flow proximal to the fluid-tight seal 250 within the housing 109 is prevented.

Alternatively, the fluid-tight seal 250 may be any means capable of allowing the advancement or withdrawal of an elongated member, while maintaining a seal around the elongated member to prevent fluid flow outside of the introducer port 110, such as washers, silicon discs with openings of different sizes or O-rings.

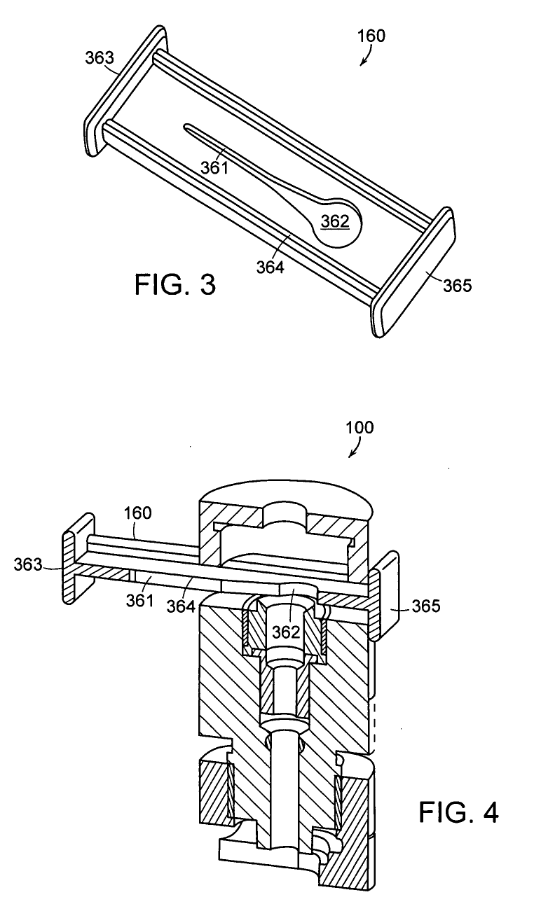

As shown in FIG. 2, the locking mechanism 160 may be a pinch clamp. Further detail of the locking mechanism or pinch clamp 160 is shown in FIGS. 3 and 4. As shown in FIG. 3, the pinch clamp 160 contains a circular opening, 362 which is initially positioned within the interior portion of the housing 109 so that it aligns with the lumen 240 and the device port 130. The diameter of the circular opening 362 is large enough, so that the elongated member can be slidably disposed within it. The pinch clamp 160 further contains a narrower opening 361. Both openings 361 and 362 are disposed on a rigid plate 364. The plate 364 may be composed of any suitable rigid material such as metal, plastic, polymer and may be formed by injection molding.

The pinch clamp 160 also includes two distinct pusher plates, 363 and 365, which may be manipulated to position the pinch clamp 160 with respect to the endoscope accessory 100. Pusher plates 363 and 365 are both exterior to the housing 109, while the plate 364 is capable of being positioned within the interior of the endoscope accessory 100.

As shown by the cross section in FIG. 4, the pinch clamp 160 extends through the interior of the endoscope accessory 100 with plates 363 and 365, and a portion of the plate 364 extending out of the exterior of the endoscope accessory 100. When the operator wishes to secure the elongated member with respect to the endoscope, the pusher plate 363 is pushed or the pusher plate 365 is pulled through the interior such that the elongated member is pinched by a narrower opening 361 of the pinch clamp 160.

If the operator wishes to unlock the elongated member, the pusher plates 363 and/or 365 are manipulated so that as opposed to the narrower opening 361, the circular opening 362 surrounds the elongated member. The elongated member is therefore, no longer pinched by the narrower opening 361 and is again slidably disposed within the circular opening 362. Alternatively, the locking mechanism 160 may be a compression type lock such as a wedge, a screw type lock or any other suitable locking device capable of securing the elongated member with respect to the endoscope accessory 100.

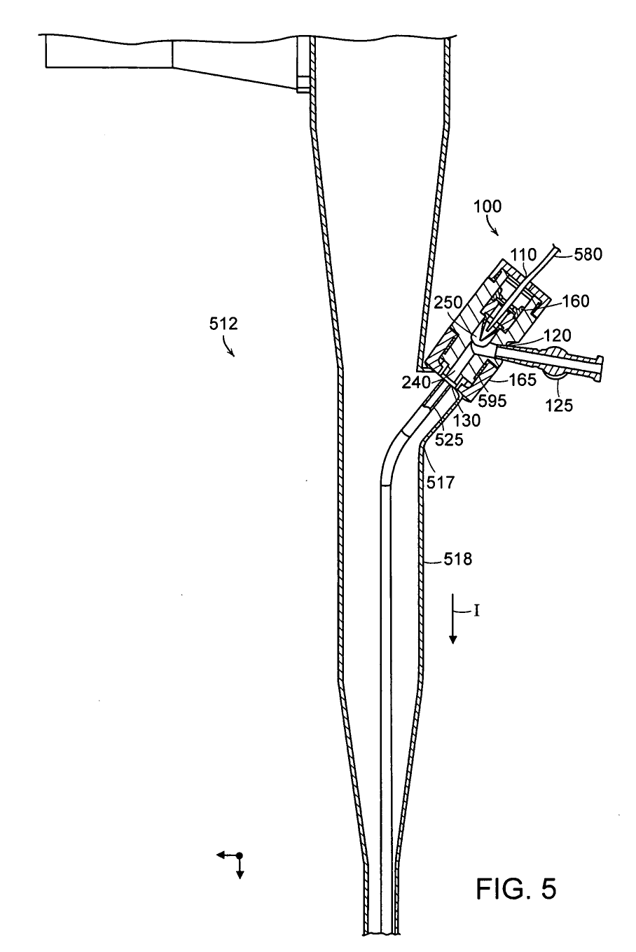

Further detail of using an endoscope accessory 100 during a medical procedure is shown by the cross section of the medical device in FIG. 5. The endoscope accessory 100 is coupled to an accessory port 517 of an endoscope 512, using the coupling mechanism 165. A lumen 525 of the accessory port 517 extends into an accessory channel 518 of the endoscope 512. As shown in this FIG. 5, the device port 130 is aligned with the lumen 525 of the accessory port 517. The elongated member 580 is extended through the accessory port 517 and extends into the accessory channel 518 of the endoscope.

As an elongated member 580 is advanced through the lumen 240, it encounters the fluid-tight seal 250. The fluid-tight seal 250 allows the elongated member 580 to pass through it and forms a seal around the elongated member 580. Alternatively, if the elongated member 580 is withdrawn from the endoscope accessory 100, by being pulled through the device port 130 and the introducer port 110, the fluid-tight seal 250, would maintain the seal around the elongated member 580.

As the elongated member 580 is being advanced through the lumen 240 during a medical procedure, the operator may choose to irrigate the patient's lumen to facilitate advancement of the elongated member 580 into the patient's body. The fluid injected through the fluid port 120 flows into the portion of lumen 240 distal to the fluid-tight seal 250, and continues to flow further into the accessory channel 518 of the endoscope 512, finally flowing into the patient's lumen as indicated by Arrow I. The fluid flow may be controlled by the valve 125.

The endoscope accessory 100 may be used in an endoscopic procedure, for example, in the placement or removal of a gastrointestinal sleeve within a patient's gastointestinal tract as described in FIG. 2. The elongated member 580 is thus advanced through the introducer port 110, the lumen 240, and the device port 130, and extends into the accessory port 517, where it continues to be advanced through the accessory channel 518 of the endoscope. The endoscope ultimately guides the elongated member 580 within the patient's gastrointestinal tract. In a repositioning or removal process of a gastrointestinal device, for example, the elongated member 580 may grasp a proximal portion of the implant.

Once the elongated member 580, has grasped the implant, the operator or endoscopist may wish to secure the elongated member 580 with respect to the endoscope 512. This would allow the endoscope, the elongated member 580 disposed therein, and the grasped implant to be removed together as a single unit from the patient's body. This is particularly advantageous in retrieving or repositioning a gastrointestinal implant, including an anchor, and especially when the device includes sharp external features, such as barbs. Examples of implantable devices are described in U.S. patent application Ser. No. 10/726,001, filed on Dec. 2, 2003, and claiming the benefit of U.S. Provisional Application No. 60/512,145, filed on Oct. 17, 2003, and incorporated herein by reference in their entireties. In addition to providing access to the patient's lumen, the endoscope 512 may also be used to view and guide the endoscopic procedure. Images from the procedure are transferred through a camera on the proximal end of the endoscope 512 to a monitor.

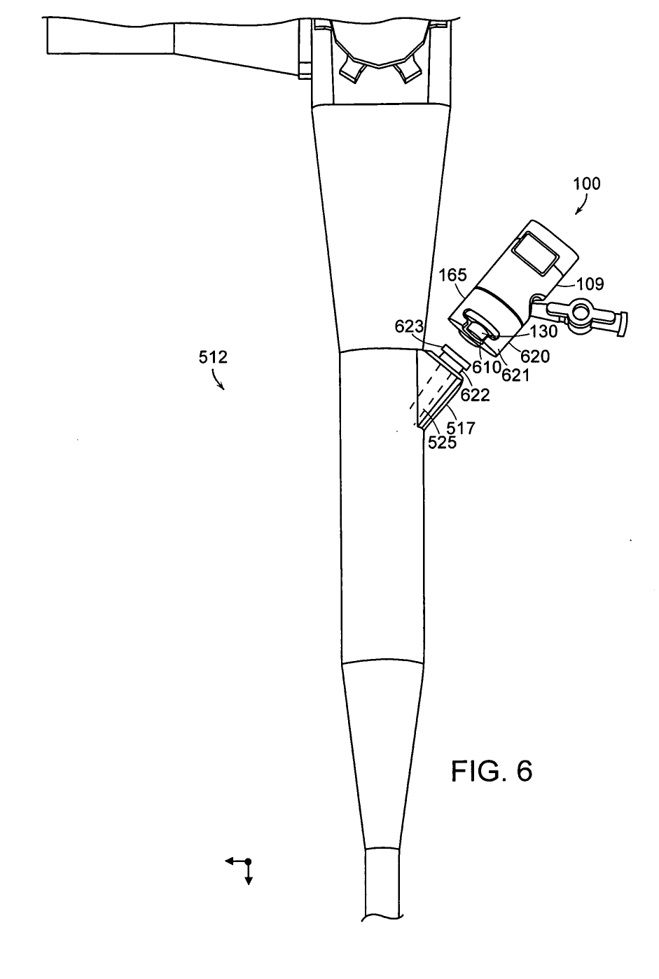

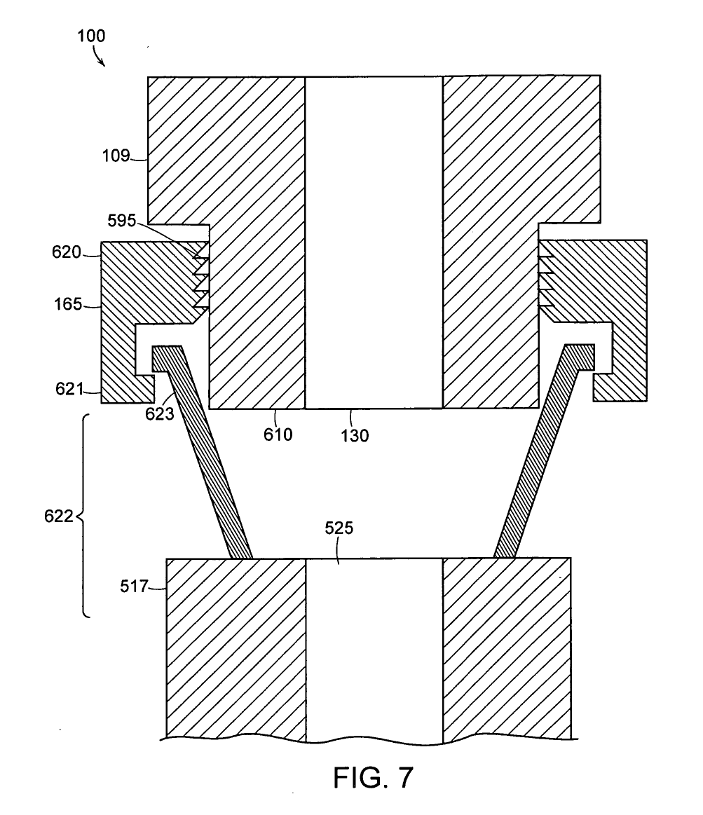

FIG. 6 illustrates a perspective view of the endoscope accessory 100 prior to coupling to the endoscope 512. In operation, the coupling mechanism 165 in FIG. 7 is loosened sufficiently on threads 595 with respect to the housing 109, such that the nipple 610 containing the device port 130 is recessed at least partially within the coupling mechanism 165. When the shoulder 621 engages the distal face of the flange 623, the coupling mechanism 165 remains coupled to the attachment fitting 622 when pulled axially.

When captured in this manner, the housing 109 is tightened with respect to the coupling mechanism 165 such that the nipple 610 advances at least partially within the lumen 525 of the attachment fitting 622. The alignment of the nipple 610 within the lumen 525 prevents lateral displacement of the endoscope accessory 100 and the endoscope 512. The tightening of the housing 109 with respect to the coupling mechanism 165, preferably provides a seal prohibiting fluid leakage from the endoscope accessory 100, while the tapering of the shoulder 621 with the flange 623 inhibits axial displacement and also provides a fluid seal. Thus, the shoulder 621 of the coupling mechanism 165, by mating with the distal face of the flange 623, adapts to the outer diameter of the attachment fitting 622 and creates a fluid seal. The tightening of the housing 109 and the fit formed by the nipple 610 within the attachment fitting 622, forms a fluid seal by adapting to the inner diameter of the attachment fitting 622. Alternatively, a fluid seal may be created by inserting a gasket, such as an O-ring between the flange 623 and the sleeve 620, as opposed to tightening the housing 109 with respect to the coupling mechanism 165

In a further embodiment of the endoscope accessory, various types of elongated members may be inserted through the endoscope accessory. For example, the elongated member may be a hook, a spade, a retrieval bag, a multi-prong grasper, a rat-tooth type grasper or any means capable of grasping onto a medical device.

In an alternative embodiment of the invention, the endoscope accessory may be coupled to a catheter, as opposed to the accessory port of an endoscope. In this case, the previously described coupling mechanism may be replaced with a Luer-type fitting, which is capable of coupling onto the proximal end of a catheter. The elongated member, would therefore, be inserted through the endoscope accessory and would extend into the catheter, and further into the patient's lumen in order to perform the medical procedure.

While this invention has been particularly shown and described with references to preferred embodiments thereof, it will be understood by those skilled in the art that various changes in form and details may be made therein without departing from the scope of the invention encompassed by the appended claims.

What is claimed is:

1. An endoscope accessory comprising:
a housing defining a lumen between a proximal introducer port and a distal device port, the lumen extending through the introducer port and the device port;
a coupling mechanism coupled to the housing, adapted to couple the device port to a proximal portion of a medical instrument;
a normally closed valve, adapted to form a seal around an elongated member inserted through the lumen, and to prohibit flow of fluid through the introducer port of the housing;
a locking mechanism coupled to the housing, adapted to secure the elongated member against axial movement with respect to the housing; and
a fluid port in fluid communication with the lumen, adapted to permit the injection of fluid through it to the device port.

2. The endoscope accessory of claim 1, wherein the medical instrument is an endoscope.

3. The endoscope accessory of claim 1, wherein the coupling mechanism is adapted to draw the device port into sealed engagement with a port of the medical instrument.

4. The endoscope accessory of claim 1, wherein the coupling mechanism is threaded on to the housing.

5. The coupling mechanism of claim 1, wherein the medical instrument is a catheter.

6. The endoscope accessory of claim 1, wherein the housing further comprises a valve adapted to regulate fluid flow through the fluid port.

7. The endoscope accessory of claim 1, wherein the locking mechanism is a pinch clamp.

8. The endoscope accessory of claim 1, wherein the normally closed valve is a duck-bill valve.

9. The endoscope accessory of claim 1, wherein the normally closed valve is an elastomer.

10. An endoscope accessory comprising:
a housing defining a lumen between a proximal introducer port and a distal device port, the lumen extending through the introducer port and the device port, the device port adapted to be coupled to a medical instrument port;
a normally closed valve, adapted to form a seal around an elongated member inserted through the lumen, and to prohibit flow of fluid through the introducer port of the housing;
a coupling mechanism adapted to couple the housing to the accessory port of an endoscope, the coupling mechanism comprising a sleeve having a shoulder to abut a distal face of an endoscope port flange toward the device port to bring the device port and an endoscope port into sealed engagement; and
a fluid port in fluid communication with the lumen, adapted to permit the injection of fluid through it to the device port.

11. The endoscope accessory of claim 10, further comprising a locking mechanism coupled to the housing, adapted to secure the elongated member against axial movement with respect to the housing.

12. The endoscope accessory of claim 11, wherein the locking mechanism is a pinch clamp.

13. The endoscope accessory of claim 10, wherein the housing further comprises a valve adapted to regulate fluid flow through the fluid port.

14. The endoscope accessory of claim 10, wherein the coupling mechanism is threaded on to the housing.

15. The endoscope accessory of claim 10, wherein the normally closed valve is a duck-bill valve.

16. The endoscope accessory of claim 10, wherein the normally closed valve is an elastomer.

17. A method of performing an endoscopic procedure, comprising the steps of:
coupling an endoscope accessory to an accessory port of an endoscope;
inserting an elongated member through the endoscope accessory into the endoscope;
irrigating a lumen in a patient's body injected through the accessory port while the elongated member is inserted therein; and
securing the elongated member against axial movement with respect to the endoscope accessory using a locking mechanism coupled to the endoscope accessory.

18. The method of claim 17, wherein coupling the endoscope accessory, further comprises coupling the endoscope accessory to the endoscope using a coupling mechanism.

19. The method of claim 18, wherein the coupling mechanism comprises a sleeve having a shoulder to abut a proximal flange on the accessory port of the endoscope.

20. The method of claim 17, wherein inserting the elongated member further comprises inserting the elongated member into an introducer port of the endoscope accessory.

21. The method of claim 17, further comprising removing the endoscope and the elongated member secured thereto from the patient's body.

22. The method of claim 17, wherein irrigating further comprises irrigating the patient's lumen as the elongated member is advanced within the patient's lumen.

23. The method of claim 17, wherein the locking mechanism is a pinch clamp.
